(19) United States
(12) Patent Application Publication
(10) Pub. No.: US 2022/0313551 A1
(43) Pub. Date: Oct. 6, 2022

(54) Bio-signal Measurement Device for Massage Apparatus

(71) Applicant: BODYFRIEND CO., LTD., SEOUL (KR)

(72) Inventors: Jong Sick PARK, Seoul (KR); Sung Gu LIM, Seoul (KR); Myeong Jin YU, Seoul (KR); Deok Hyun KONG, Seoul (KR); Young Hoon CHO, Seoul (KR)

(21) Appl. No.: 17/714,626

(22) Filed: Apr. 6, 2022

(30) Foreign Application Priority Data
Apr. 6, 2021 (KR) 10-2021-0044836
May 27, 2021 (KR) 10-2021-0068375

(51) Int. Cl. A61H 39/04

(52) U.S. Cl. CPC A61H 39/04; A61H 2201/1666; A61H 2201/1215; A61H 2201/5064; A61H 2205/12

(57) ABSTRACT

Provided is a biometric signal measurement device for a massage apparatus, in which an electrode for acquiring a biometric signal of a user acquires the biometric signal while vertically moving in contact with a plantar part of the user, and at the same time, massages the plantar part, thereby improving a massage effect. The biometric signal measurement device for a massage apparatus includes a base part on which a foot of a user is placed, an electrode part that is made of a conductive material, is installed in the base part to be vertically movable, and acquires a biometric signal while in contact with a sole of the user placed on the base part, an electrode driving part that vertically moves the electrode part, and a controller that is electrically connected to the electrode part and analyzes biometric information of the user through the biometric signal acquired by the electrode part.

### CROSS-REFERENCE TO RELATED APPLICATION

This application claims priority to and the benefit of Korean Patent Application Nos. 10-2021-0044836 filed on Apr. 6, 2021, and 10-2021-0068375 filed on May 27, 2021, the disclosures of which are incorporated herein by reference in their entirety.

### BACKGROUND

### 1. Field of the Invention

The present disclosure relates to a massage apparatus, and more particularly, to a biometric signal measurement device for a massage apparatus, which measures a biometric signal while in contact with a plantar part, particularly, a heel part, of a user.

### 2. Discussion of Related Art

In recent years, with the aging of society, awareness of health management, health maintenance, and health promotion has been increasing among people of all ages. In general, many people not only receive diagnosis or advice of experts from medical facilities such as hospitals for the purpose of early detection or prevention of diseases but also try to manage, maintain or improve their own health by using non-medical facilities such as so-called sports clubs.

Further, apparatuses used by people to maintain or improve their health and physical strength or relieve fatigue and stress at home or at a gym, for example, exercise machines such as treadmills or massage machines, are widely used in daily life.

A massage is a type of auxiliary medical therapy that helps blood circulation and relieves fatigue by sweeping, kneading, pressing, pulling, tapping, or moving the body with hands or special tools. An apparatus for performing a massage with a mechanical device is called a massage apparatus, and various mechanical elements are used in such a massage apparatus for an effective massage.

One such massage apparatus is a massage chair in which a user may receive a massage while sitting comfortably.

A general massage chair includes a main body that supports a body of a user, and the main body includes a hip/thigh support that supports the hips and thighs of the user, an upper body support that is rotatably installed on a rear end side of the hip/thigh support and supports the upper body of the user, and a calf/foot support that is rotatably installed on a front end side of the hip/thigh support and supports the calves and feet of the user.

In recent years, various functions that allow users to rest more comfortably or to relieve fatigue more effectively in addition to a function of performing a massage are being added to massage chairs.

However, health care services that are considered in development of all the functions being added to massage chairs are limited to those that are possible through measurement of physical characteristics, and therefore users simply receive massages for physical health management and do not receive health care based on other biometric characteristics.

In order to solve this problem, Korean Patent Registration No. 10-1879209 discloses a massage chair that may detect health indexes such as a blood pressure, a stress level, a tension level, and a blood vessel age using biometric signals measured by a sensor unit.

However, biometric signal measurement devices configured in massage apparatuses according to the related art as well as the massage chair according to the registered patent perform only a function of acquiring a biometric signal while an electrode is simply in close contact with the body and cannot provide a massage effect by applying acupressure while the electrode is in close contact with the body.

### SUMMARY OF THE INVENTION

The present disclosure is directed to providing a biometric signal measurement device for a massage apparatus, in which an electrode for acquiring a biometric signal of a user acquires the biometric signal while vertically moving in contact with a plantar part of the user, and at the same time, massages the plantar part, thereby improving a massage effect.

A biometric signal measurement device for a massage apparatus includes a base part on which a foot of a user is placed, an electrode part that is made of a conductive material, is installed in the base part to be vertically movable, and acquires a biometric signal while in contact with a sole of the user placed on the base part, an electrode driving part that vertically moves the electrode part, and a controller that is electrically connected to the electrode part and analyzes biometric information of the user through the biometric signal acquired by the electrode part.

The electrode part may include an electrode body having a flat plate shape and an acupres sure electrode protruding upward from the electrode body to stimulate the sole of the user.

The electrode driving part may include a driving motor that operates by receiving a control signal from the controller, a driving shaft that rotates by receiving power from the driving motor, an eccentric cam coupled to the driving shaft to rotate eccentrically, a cam housing which has one end rotatably connected to the base part via a hinge shaft, has a cam accommodation hole into which the eccentric cam is inserted and coupled, and oscillates in an up-down direction and a front-rear direction with respect to the hinge shaft due to the eccentric rotation of the eccentric cam, and a mount bracket installed at an upper end of the cam housing to support the electrode part.

An acupressure boss that stimulates the sole of the user may be formed on the mount bracket to protrude upward.

A guide bracket for coupling the electrode part to an upper side of the mount bracket may be installed between the mount bracket and the electrode part.

A hinge guide hole which has a slot shape and to which the hinge shaft is insertion-connected may be formed at a lower end of the cam housing to pass through the cam housing in a lateral direction.

The electrode driving part may further include a location sensing unit that detects whether the electrode part reaches a top dead point when the electrode part moves vertically. The location sensing unit may include a sensor dog that is coupled to the driving shaft and rotates together with the driving shaft, and a location sensor that detects a location of the sensor dog at a time point at which the cam housing reaches the top dead point.

The controller may analyze the biometric information based on the biometric signal acquired from the electrode part when the electrode part reaches the top dead point.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a perspective view illustrating a foot massage part of a massage chair to which a biometric signal measurement device for a massage apparatus according to an embodiment of the present disclosure;

FIG. 2 is a plan view illustrating a main part of the foot massage part illustrated in FIG. 1;

FIG. 3 is a perspective view illustrating the biometric signal measurement device for a massage apparatus according to an embodiment of the present disclosure;

FIG. 4 is an exploded perspective view of the biometric signal measurement device for a massage apparatus illustrated in FIG. 3;

FIG. 5 is a bottom perspective view illustrating a part of the biometric signal measurement device for a massage apparatus illustrated in FIG. 3;

FIG. 6 is a side view of the biometric signal measurement device for a massage apparatus illustrated in FIG. 3;

FIGS. 7A and 7B are a side view illustrating an operation example of the biometric signal measurement device for a massage apparatus illustrated in FIG. 3 and a perspective view of a location sensing unit;

FIG. 8 is a perspective view illustrating a biometric signal measurement device for a massage apparatus according to another embodiment of the present disclosure;

FIG. 9 is a perspective view illustrating a part of the biometric signal measurement device for a massage apparatus illustrated in FIG. 8; and

FIG. 10 is a perspective view illustrating a biometric signal measurement device for a massage apparatus according to still another embodiment of the present disclosure.

### DETAILED DESCRIPTION OF EXEMPLARY EMBODIMENTS

Other purposes, characteristics, and advantages of the present disclosure will become apparent from a detailed description of embodiments with reference to the accompanying drawings.

Exemplary embodiments of a biometric signal measurement device for a massage apparatus according to the present disclosure will be described below with reference to the accompanying drawings. However, the present disclosure is not limited to the embodiments disclosed below but may be implemented in various different forms, and the present embodiment is merely intended to complete the present disclosure and is provided to completely inform those skilled in the art of the scope of the present disclosure. Thus, since the embodiments described in the present specification and configurations illustrated in the drawings are merely the most exemplary embodiments of the present disclosure and do not represent all the technical spirit of the present disclosure, it should be understood that there exist various equivalents and variations that may replace the embodiments and the configurations at the time of filing of the present application.

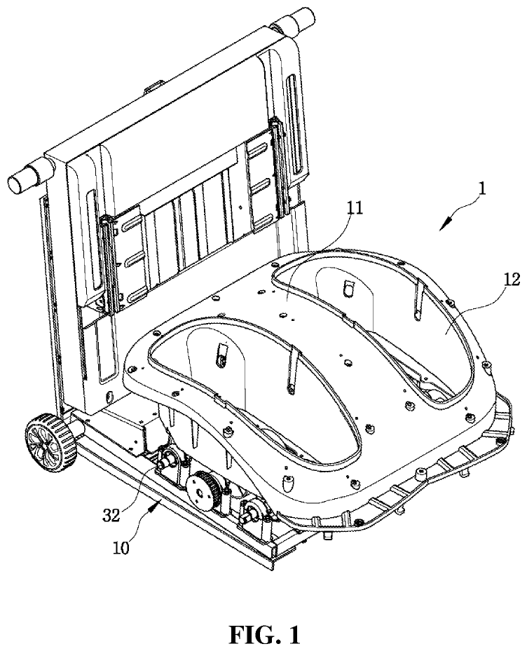

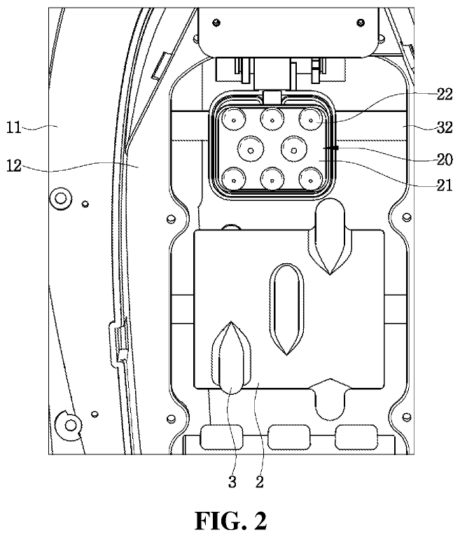

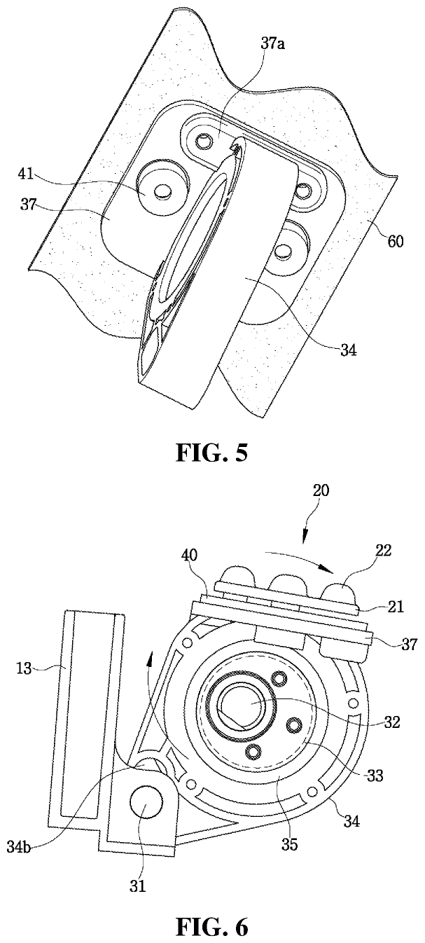

In FIGS. 1 to 7B, the biometric signal measurement device for a massage apparatus according to an embodiment of the present disclosure includes a base part 10 on which a foot of a user is placed, an electrode part 20 which is made of a conductive material, is installed in the base part 10 to be vertically movable, and acquires a biometric signal while in contact with a sole of the user placed on the base part 10, an electrode driving part that vertically moves the electrode part 20, and a controller (not illustrated) that is electrically connected to the electrode part 20 and analyzes biometric information of the user through the biometric signal acquired by the electrode part 20.

The base part 10 is made to support the foot of the user in a foot massage part 1 of the massage apparatus such as a massage chair, and a foot guide member 11 formed so that a foot accommodation groove 12 into which the foot of the user is inserted is vertically opened may be installed in the base part 10. Further, a plantar massage unit for massaging the sole of the user is configured under the foot guide member 11 of the base part 10. In the present embodiment, the plantar massage unit may include a cylindrical massage member 2 that rotates by receiving a rotational force from a motor (not illustrated) and a plurality of massage bosses 3 formed to protrude from an outer surface of the massage member 2. In addition, the plantar massage unit may be configured by applying a widely known massage unit configured in the foot massage part 1 of a general massage chair to massage the sole.

The base part 10 is covered with a covering material 60, such as cloth, leather, artificial leather, or a resin so that various components configured in the base part 10 are concealed, and the user may feel a smooth sensation and comfort upon inserting the foot into the foot accommodation groove 12 of the foot guide member 11 of the base part 10.

The electrode part 20 is disposed below the foot accommodation groove 12 of the foot guide member 11 and acquires the biometric signal while in contact with the sole of the user when the user has inserted the foot into the foot accommodation groove 12 of the foot guide member 11. The electrode part 20 may be made of a conductive metal material having excellent electrical conductivity, such as copper, or may be made in the form of a flat plate plated with a conductive metal. One side of the electrode part 20 is connected to the controller (not illustrate) through an electrical wire.

The electrode part 20 is configured to acquire the biometric signal while in contact with the sole, particularly, a heel part, of the user, and at the same time, to perform a massage action of pressing the sole while being moved vertically by the electrode driving part. In order to improve a sole massaging effect of the electrode part 20, at least one acupressure electrode 22 may be formed to protrude from a surface of the electrode part 20. For example, as in the present embodiment, the electrode part 20 includes a flat electrode body 21 and a plurality of acupressure electrodes 22 protruding upward from the electrode body 21 to stimulate the sole, and while moving vertically, the electrode body 21 and the acupressure electrodes 22 may stimulate the sole of the user through acupres sure.

Further, the electrode part 20 is installed outside the covering material 60 covering the base part 10 so that the electrode part 20 acquires the biometric signal while in direct contact with the sole of the user.

The electrode driving part for vertically moving the electrode part 20 includes a driving motor (not illustrated) that operates by receiving a control signal from the controller (not illustrated), a driving shaft 32 that rotates by receiving power from the driving motor (not illustrated), an eccentric cam 33 coupled to the driving shaft 32 to rotate eccentrically, a cam housing 34 which has one end rotatably connected to a support bracket 13 of the base part 10 via a hinge shaft 31 and is oscillated in an up-down direction and a front-rear direction by the eccentric cam 33, and a mount bracket 37 installed at an upper end of the cam housing 34 to support the electrode part 20.

The driving motor (not illustrated) is installed in the base part 10, an operation of the driving motor is controlled by the controller, and the driving motor is directly connected to the driving shaft 32 or is connected to the driving shaft 32 via a power transmission mechanism such as a gear or a pulley to rotate the driving shaft 32.

The driving shaft 32 has one end rotatably supported on the base part 10 through a bearing (not illustrated) and rotates by receiving the power from the driving motor (not illustrated).

The eccentric cam 33 is eccentrically coupled to an outer surface of the driving shaft 32 and rotates together with the driving shaft 32 to oscillate the cam housing 34 in the up-down direction and the front-rear direction. The eccentric cam 33 may be installed inside a cam accommodation hole 34a formed in the cam housing 34 and a bearing 35 may be installed between an outer surface of the eccentric cam 33 and an inner surface of the cam accommodation hole 34a. Further, a separation prevention washer 36 for preventing separation between the eccentric cam 33 and the bearing is coupled to one side of the eccentric cam 33.

The cam housing 34 is formed so that the circular cam accommodation hole 34a into which the eccentric cam 33 and the bearing 35 are inserted and coupled is open. Further, a hinge guide hole 34b which has a slot shape extending vertically and into which the hinge shaft 31 for connecting the cam housing 34 to the support bracket 13 is inserted is formed on one side of a lower end of the cam housing 34 to pass through the cam housing 34 in a lateral direction.

Thus, when the eccentric cam 33 eccentrically rotates inside the cam accommodation hole 34a of the cam housing 34 due to the rotation of the driving shaft 32, the cam housing 34 oscillates in the up-down direction and the front-rear direction with respect to the hinge shaft 31.

The mount bracket 37 for installing the electrode part 20 is installed at an upper end of the cam housing 34. The mount bracket 37 may be made integrally with the cam housing 34, or may be made separately from the cam housing 34 and then fixedly coupled to an upper surface of the cam housing 34 through a fastening member such as a screw.

Further, since the mount bracket 37 is disposed below the covering material 60 and the electrode part 20 is disposed above the covering material 60, it may be difficult to directly assemble the electrode part 20 to the mount bracket 37. Accordingly, a guide bracket 40 for coupling the electrode part 20 to an upper side of the mount bracket 37 may be installed between the mount bracket 37 and the electrode part 20. In this case, in a state in which the covering material 60 is interposed between the electrode part 20 and the guide bracket 40, a fastening boss 23 of the electrode part 20 is inserted into a second fastening boss 41 of the guide bracket 40 through a hole 61 of the covering material 60, and the fastening boss 23 is coupled to the second fastening boss 41 through a fastening member (not illustrated) such as a bolt or a screw. Thereafter, a third fastening boss 42 of the guide bracket 40 to which the electrode part 20 is assembled is aligned with a fourth fastening boss 37a of the mount bracket 37, and the third fastening boss 42 is coupled to the fourth fastening boss 37a through a fastening member (not illustrated) such as a bolt or a screw. Thus, the electrode part 20 may be easily assembled on the mount bracket 37.

Meanwhile, when the biometric signal of the user is acquired through the electrode part 20 while the electrode part 20 moves vertically, the biometric signal is preferably acquired when a contact pressure between the electrode part 20 and the sole of the user is greatest, that is, when the electrode part 20 reaches a top dead point, in order to improve the accuracy. Accordingly, a location sensing unit that detects whether the electrode part 20 reaches the top dead point is configured.

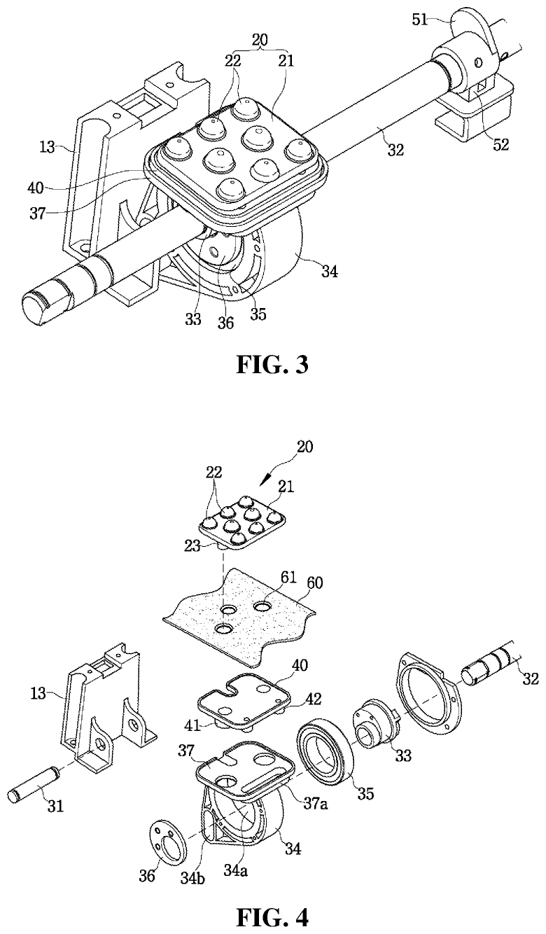

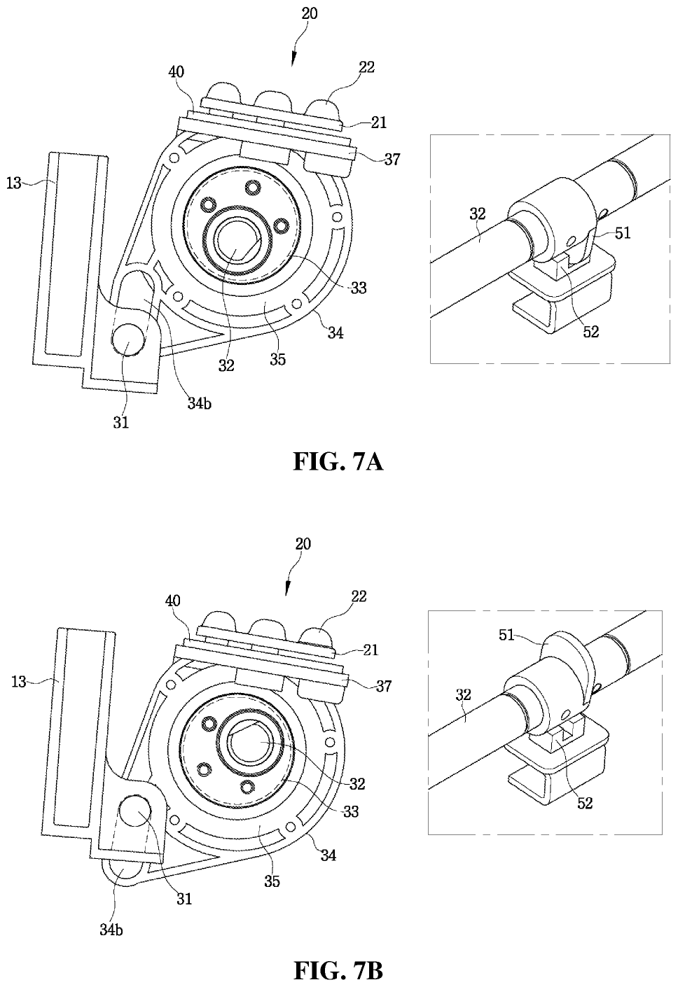

As illustrated in FIGS. 3, 7A, and 7B, the location sensing unit includes a sensor dog 51 that is coupled to the driving shaft 32 and rotates together with the driving shaft 32 and a location sensor 52 that detects a location of the sensor dog 51 at a time point at which the cam housing 34 reaches the top dead point.

The sensor dog 451 is installed to protrude radially from the outer surface of the driving shaft 32, and the location sensor 52 is installed in the base part 10, includes a light emitting unit that emits light and a light reception unit on which the emitted light is incident, and detects the sensor dog 51 whenever the sensor dog 51 rotates once.

The controller (not illustrated) may be configured as an electric component that controls operation of all components of the massage chair and analyzes biometric information of the user on the basis of the biometric signal acquired from the electrode part 20 when the electrode part 20 reaches the top dead point while the electrode part 20 moves vertically.

The biometric information acquired by the controller may include a body composition or an electrocardiogram. The body composition is information on components constituting the body. For example, the body composition may include information on intracellular water, extracellular water, body water, protein, minerals, body fat, muscle mass, body fat mass, skeletal muscle mass, body fat percentage, body mass index (BMI), region-specific muscle mass, region-specific body water, anasarca, region-specific edema, body cell mass, bone mineral content, abdominal fat rate, visceral fat cross-sectional area, and basal metabolism rate, and the present disclosure is not limited thereto.

When the controller allows a weak alternating current (AC) current to flow to the human body through the electrode part 20, the current flows through the highly conductive body water, and a width of a passage through which the electricity flows is determined according to the amount of water. This is expressed as a measured value of an impedance, and the controller may calculate the body composition using the measured impedance.

The electrocardiogram may be a record of the electrical activity of the heart. The electrocardiogram may be recorded with an ammeter by inducing an active current generated in the myocardium according to beating of the heart to two appropriate electrode parts 20 on a body surface. The electrocardiogram may be used not only to measure the rate and consistency of the beating of the heart but also to determine the size and location of the heart and whether there is any damage to the heart.

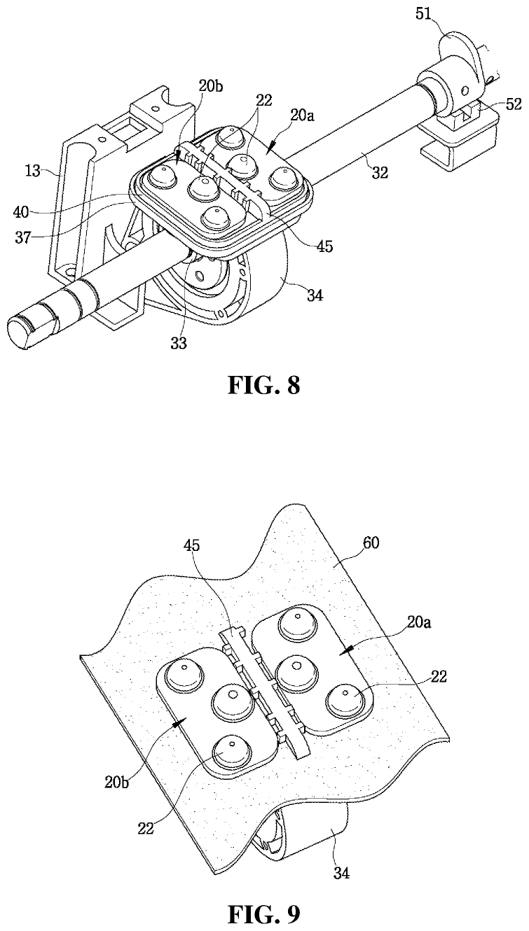

The controller may measure the body composition in a two-pole electrode method or a four-pole electrode method using at least two electrode parts 20. To this end, as illustrated in FIGS. 8 and 9, the electrode part 20 may be divided into a first electrode part 20a and a second electrode part 20b as a plurality. In this case, a partition wall part 45 separating the first electrode part 20a and the second electrode part 20b may be formed to protrude upward from an upper surface of the guide bracket 40. The partition wall part 45 protrudes outward from the upper side of the covering material 60 to separate the first electrode part 20a and the second electrode part 20b.

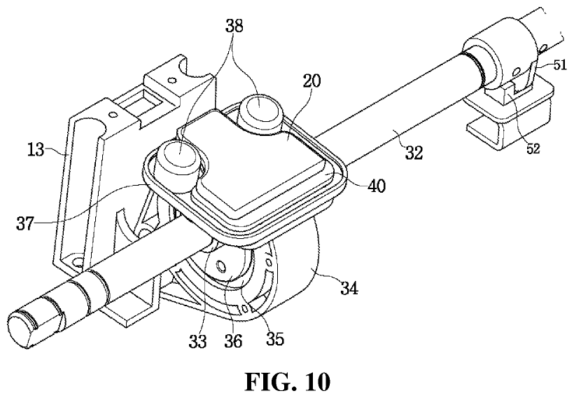

Meanwhile, in the above-described embodiment, the plurality of acupressure electrodes 22 for stimulating the sole are formed to protrude from the electrode part 20. However, as illustrated in FIG. 10, a plurality of acupressure bosses 38 for stimulating the sole of the user may also be formed to protrude upward from the mount bracket 37.

According to the present disclosure, since an electrode part electrically connected to a controller presses a sole, particularly, a heel part, of a user while being caused to periodically reciprocate vertically by an electrode driving part, the sole can be massaged through acupressure while a biometric signal is acquired by the electrode part.

In particular, the electrode part is securely in contact with the sole of the user while moving vertically, biometric information is analyzed by acquiring the biometric signal at a top dead point at which the electrode part is in contact with the sole at the greatest pressure, and thus accurate analysis can be performed.

Although the technical spirit of the present disclosure has been described above in detail in the exemplary embodiment, it should be noted that the above embodiment is intended to describe the technical spirit and not to limit the technical spirit. Further, those skilled in the art in the technical field of the present disclosure will understand that various embodiments are possible within the scope of the technical spirit of the present disclosure. Thus, the technical protection scope of the present disclosure will be defined by the technical spirit of the appended claims.

What is claimed is:

1. A biometric signal measurement device for a massage apparatus, comprising:
a base part on which a foot of a user is placed;
an electrode part that is made of a conductive material, is installed in the base part to be vertically movable, and acquires a biometric signal while in contact with a sole of the user placed on the base part;
an electrode driving part that vertically moves the electrode part; and
a controller that is electrically connected to the electrode part and analyzes biometric information of the user through the biometric signal acquired by the electrode part.

2. The biometric signal measurement device of claim 1, wherein the electrode part includes an electrode body having a flat plate shape and an acupres sure electrode protruding upward from the electrode body to stimulate the sole of the user.

3. The biometric signal measurement device of claim 1, wherein the electrode driving part includes:
a driving motor that operates by receiving a control signal from the controller;
a driving shaft that rotates by receiving power from the driving motor;
an eccentric cam coupled to the driving shaft to rotate eccentrically;
a cam housing which has one end rotatably connected to the base part via a hinge shaft, has a cam accommodation hole into which the eccentric cam is inserted and coupled, and oscillates in an up-down direction and a front-rear direction with respect to the hinge shaft due to the eccentric rotation of the eccentric cam; and
a mount bracket installed at an upper end of the cam housing to support the electrode part.

4. The biometric signal measurement device of claim 3, wherein an acupressure boss that stimulates the sole of the user is formed on the mount bracket to protrude upward.

5. The biometric signal measurement device of claim 3, wherein a guide bracket for coupling the electrode part to an upper side of the mount bracket is installed between the mount bracket and the electrode part.

6. The biometric signal measurement device of claim 3, wherein a hinge guide hole which has a slot shape and to which the hinge shaft is insertion-connected is formed at a lower end of the cam housing to pass through the cam housing in a lateral direction.

7. The biometric signal measurement device of claim 3, wherein the electrode driving part further includes a location sensing unit that detects whether the electrode part reaches a top dead point when the electrode part moves vertically.

8. The biometric signal measurement device of claim 7, wherein the location sensing unit includes:
a sensor dog that is coupled to the driving shaft and rotates together with the driving shaft; and
a location sensor that detects a location of the sensor dog at a time point at which the cam housing reaches the top dead point.

9. The biometric signal measurement device of claim 7, wherein the controller analyzes the biometric information based on the biometric signal acquired from the electrode part when the electrode part reaches the top dead point.
